(19) United States
(12) Patent Application Publication
(10) Pub. No.: US 2024/0368508 A1
(43) Pub. Date: Nov. 7, 2024

(54) STACKABLE PLATES FOR CULTURING TISSUE MODELS

(71) Applicant: MATTEK CORPORATION, Ashland, MA (US)

(72) Inventors: Mitchell Klausner, Ashland, MA (US); Seyoum Ayehunie, Ashland, MA (US); Paul Kearney, Ashland, MA (US); Alex Armento, Ashland, MA (US)

(21) Appl. No.: 18/573,293

(22) PCT Filed: Jun. 27, 2022
(86) PCT No.: PCT/US2022/035041

§ 371 Date: Dec. 21, 2023

Related U.S. Application Data
(60) Provisional application No. 63/215,892, filed on Jun. 28, 2021.
(60) Provisional application No. 63/215,895, filed on Jun. 28, 2021.

(51) Int. Cl. C12M 1/32; C12M 1/00; C12M 1/12; C12M 3/00

(52) U.S. Cl. CPC C12M 23/12; C12M 21/08; C12M 23/30; C12M 25/04

(57) ABSTRACT

Stackable tissue culture supports, such as stackable multi-well plates with membrane bottoms are provided, which permit co-culturing of multiple physiological tissues in proximity, and are used for high throughput analysis. A biodegradable membrane may be included, which allows cell to cell contact between the different cell types/physiological tissues as the membrane degrades.

### CROSS-REFERENCE TO RELATED APPLICATIONS

This application claims the benefit of U.S. Provisional Application Nos. 63/215,892, filed on Jun. 28, 2021, and 63/215,895, filed on Jun. 28, 2021, both of which are incorporated by reference herein in their entireties.

### FIELD OF THE INVENTION

The invention relates to nested support plates for coculturing of multiple two-or three-dimensional tissue culture models in a stackable, interconnected format, which allows crosstalk between physiological systems.

### BACKGROUND

In vitro tissue model systems, or “tissue equivalents,” may be used to study the effects of various agents on a variety of cell types. For example, in vitro skin equivalents may be used to test the effects of substances such as cosmetics or medications, or agents such as light and heat, for potential toxicity, irritation, or inflammation that may result from their application to skin. In comparison with in vivo animal models or studies using human test subjects, such tissue model systems offer substantial advantages in terms of reproducibility, speed of testing, and reduced cost.

For applications of use in which more than one organ system is involved, such as modeling of disease states or screening of drug candidates, it is desirable to test in a system which mimics an in vivo multi-organ environment. A device which enables construction of a tissue model which includes multiple physiological systems that are capable of interaction with one another is desirable.

### BRIEF SUMMARY OF THE INVENTION

A system of stackable tissue culture supports, such as high throughput multi-well tissue culture plates, is provided. The system described herein allows one or more microporous membrane(s), each situated at the bottom of a well that is configured to hold culture medium, to support a culture of a plurality, such as two or more, or three or more, two-dimensional or three-dimensional physiological tissue models (organotypic or organoids), such as, but not limited to, intestine-liver-immune cells, lung-liver-immune cells, or intestinal-liver-kidney. The stackable supports allow for crosstalk (intercellular communication/signaling or exchange of metabolites) between the physiological tissue systems. The stackable support systems described herein can be used to assess drug safety and efficacy, nanoparticle toxicity, fibrosis, etc. Stackable high throughput plates with tissue culture compatible membranes, as described herein, may be used to culture several types of tissue models in a high throughput format.

In some embodiments, a ridge is provided on the underside of a well of the tissue culture support. The ridge is configured to hold culture medium and may be seeded with cells to be grown on the underside of the membrane, with the well inverted (upside down) to hold cells and culture medium for growth of the cells. After the cells have adhered on the bottom side of the membrane, the well may be turned over (right side up) and other cells may be seeded on the top side of the membrane. In other embodiments, a removable sealing plate with holes that are fitted with an elastomeric material (such as, but not limited to, thermoplastic polyurethane (TPU) and thermoplastic elastomers (TPE) of appropriate Shore durometer), or a removable plate with holes that contain embedded o-rings, is used as a guide and enclosure for seeding cells and containing cells and medium on the underside (basolateral side) of membranes in wells of the tissue culture support. In these embodiments, the cell/medium mixture is seeded onto the underside of the membrane and then removed about 2 to about 24 hours later once the cells have attached to the membrane.

Methods are also provided herein for culturing tissues/cell suspensions from different physiological/pathological systems, such as, but not limited to, normal or modified immune cells and cancer cells, in a stackable, inter-connected format.

In the stackable tissue culture support systems described herein, the membranes may be: (a) inert microporous non-biodegradable membranes such as polycarbonate, polyethylene terephthalate (PET), or polytetrafluoroethylene (PTFE); (b) biodegradable, e.g., electrospun nanofiber, membranes such as polylactic-co-glycolic acid (PLGA), poly (2-cyano-acrylate) (PCA), or polycaprolactone (PCL); or (c) biological membranes such as collagen, chitosan, or glycosaminoglycan (GAG).

In one aspect, a stackable tissue culture platform is provided, comprising from top to bottom: (a) a top cover; (b) a culture support plate that comprises a plurality of wells, wherein the wells are open at the top and wherein at least a portion of the bottom of each of the wells comprises a microporous biodegradable membrane that is suitable for seeding of tissue culture cells; and (c) a liquid reservoir plate, wherein the reservoir plate comprises individual wells configured such that the wells of the culture support plate nest within the wells of the reservoir plate, or wherein the reservoir plate comprises a common area for holding liquid to which the membranes at the bottoms of all wells of the culture support plate are accessible.

In some embodiments, the stackable tissue culture support further comprises: one or more additional culture support plates, stacked above the culture support plate of (b) and beneath the top cover of (a), wherein the one or more additional culture support plates each comprise the same number and arrangement of wells as the culture support plate of (b), and wherein the wells of each additional culture support plate are configured to nest within the wells of the culture support plate that is directly beneath, and wherein the wells of each additional culture support plate are each open at the top and wherein at least a portion of the bottom of each of the wells of each additional culture support plate comprises a microporous membrane that suitable for seeding of tissue culture cells.

In some embodiments, the stackable tissue culture support comprises, from top to bottom: (a) a top cover; and (b) two or more stacked culture support plates, wherein each of the stacked culture support plates comprises the same number and arrangement of wells as each of the other culture support plates in the stack, and wherein the wells of each culture support plate are configured to nest within the wells of the culture support plate that is directly beneath, and wherein each culture support plate comprises a plurality of wells, wherein the wells are open at the top and wherein at least a portion of the bottom of each of the wells comprises a microporous membrane that is suitable for seeding of tissue culture cells; and (c) a liquid reservoir plate, wherein the reservoir plate comprises individual wells configured such that the wells of the culture support plate directly above the reservoir plate nest within the wells of the reservoir plate, or wherein the reservoir plate comprises a common area for holding liquid to which the membranes at the bottoms of all wells of the culture support plate directly above the reservoir plate are accessible.

In some embodiments, each well of the culture support plate(s) comprises an under-ridge around the bottom of the well that provides a bounded area for seeding of cells on the basolateral surface of the membrane when the plate is inverted, thereby providing an enclosure to contain cells and medium, and wherein the under-ridges are separately attached underneath each well, or wherein the under-ridges are provided as an interconnected matrix under the wells of the plate with an under-ridge attached underneath each well.

In another aspect, a removable plate is provided, comprising openings that match the geometry of the wells of a tissue culture support plate as described herein, wherein the openings comprise holes that are tapered to fit over the basolateral side of the microporous membranes when the culture support plate is inverted, and wherein the tapered openings provide a conduit for seeding of cells on the basolateral surface of the membrane and provide an enclosure to contain cells and medium, and wherein the holes are fitted with a flexible, elastomeric material that provides a seal between each opening of the removable plate and the corresponding microporous membrane in the culture support plate, thereby preventing leakage of medium when cells are seeded on the undersides of the membranes.

In another aspect, a removable plate is provided, comprising openings that match the geometry of the wells of a tissue culture support plate as described herein, wherein the o-rings are embedded in the holes such that when the removable plate is placed on top of an inverted culture support plate, the o-rings provide a guide for seeding of cells on the basolateral surfaces of the membranes and provide an enclosure to contain cells and medium.

In another aspect, a stackable tissue culture support as described herein further comprises a riser between a culture support plate and the liquid reservoir plate, and/or between stacked culture support plates, wherein the riser comprises material that is sufficient to separate the plates by a desired height, and wherein the riser comprise lengths corresponding to the lengths of the outer edges of the culture support plate and the liquid reservoir plate, and wherein the riser extends along the outer edge perimeter between the plates, thereby separating the plates by the desired height.

### BRIEF DESCRIPTION OF THE DRAWINGS

FIGS. 1A-1B show biodegradation of a PLGA electrospun membrane following storage in PBS at 37° C. at Day 0 (1A) and Day 28 (1B). By Day 28, the size of the sample had increased significantly, and the membrane had become nearly transparent.

FIGS. 2A-2B show single well, cell culture inserts (CCI) produced with a PLGA electrospun membrane and an under-ridge (height=0.9 mm), which may be used for seeding cells on the underside of the membrane. 2A: Top view of four inserts. 2B: Bottom view of sixteen inserts (inverted), showing the under-ridge.

FIGS. 3A-3C show H&E stained cross-sections of the EpiIntestinal™ tissue model (MatTek Corporation) cultured in single-well cell culture inserts (CCI) constructed with: an uncoated, PLGA membrane (3A); a PET, collagen-coated membrane (3B); and human, explant small intestine tissue (3C).

FIGS. 4A-4B show H&E stained cross-sections of the EpiIntestinal™ tissue model (MatTek Corporation) cultured in single-well cell culture inserts (CCI) with under-ridges constructed with a PLGA membrane for 13 days (4A) and 17 days (4B).

FIGS. 5A-5B show H&E stained cross-sections of the EpiIntestinal™ tissue model cultured in single-well cell culture inserts (CCI) with under-ridges constructed with a PLGA membrane for 13 days (5A) and 21 days (5B).

FIGS. 6A-6B show a molded 96-well cell culture insert plate (CCIP) prior to attachment of a PLGA membrane: inverted view showing bottoms of the wells (6A); and top view (6B).

FIGS. 7A-7B show an inverted view of a molded 96-well CCIP, after attachment of rectangular sheet (82×124 mm) of PLGA membrane (7A), and after trimming excess membrane between wells (7B).

FIGS. 8A-8B show an inverted view of a 96-well cell CCIP with PLGA membrane, after attachment of a mesh of under-rings (8A), and after trimming of inter-connecting pieces between the under-rings (8B).

FIGS. 9A-9D show an example of a stackable multi-well culture plate assembly: top cover (9A); culture plate with microporous support membrane 91 at bottoms of wells (9B); bottom reservoir plate with liquid reservoirs in individual wells (9C) or common liquid reservoir accessible by all wells of the culture plate (9D).

FIG. 10 shows a cross-section of an example of a stacked multi-well culture plate assembly, with a top cover 101, two culture cell plates 102 and 103 stacked with nested wells, and a bottom reservoir plate 104 with individual liquid reservoir wells.

FIGS. 11A-11D show examples of under-ridges for seeding cells on the bottom (basolateral) side of membranes in a stackable multi-well culture plate assembly: individual under-ridge rings (11A); inter-connected under-ridge rings (11B); inverted 96-well plate with interconnected under-ridges attached (11C); inverted 96-well plate with interconnections between under-ridges removed (11D).

FIG. 12 shows a cross-section of an example of a stacked multi-well culture plate assembly, with a top cover 121, two culture cell plates 122 and 123 stacked with nested wells, an under-ridge 125 on the bottom (basolateral side) of each well, and a bottom reservoir plate 124 with individual liquid reservoir wells.

FIGS. 13A-13C shows an example of a sealing ring plate for seeding cells on the bottom surfaces of membranes in wells of a stacked multi-well culture plate assembly. An elastomeric material is situated on the inside of holes that correspond to wells of the multi-well culture plate. 13A: Top view of plate, configured to contact basolateral side of membranes in each well of the multi-well plate. 13B: Bottom view of plate, showing tapered holes which serve as conduit for seeding cells on underside (basolateral side) of membranes. 13C: Cross-section of plate 131, with elastomeric material 132 on inside of holes. The inverted 96-well culture plate 133 fits snugly into the elastomeric material to allow seeding of cells onto the underside of membrane 134 of the 96-well culture plate when inverted.

FIGS. 14A-14C show an example of a plate with embedded o-rings for seeding cells on bottom surfaces of membranes in wells of a stacked multi-well culture plate assembly. 14A: Top view of plate, configured to contact basolateral side of membranes in each well of the multi-well plate. 14B: Cross-section, showing embedded o-rings, which form an enclosure for seeding cells on underside (basolateral side) of membranes. 14C: Bottom view of plate 141 with embedded o-rings 142 on top of inverted 96-well culture plate 143. The device allows for seeding of cells onto the underside of membrane 144 of the 96-well culture plate when inverted.

FIGS. 15A-15B shows an example of a riser that is configured to fit between the bottom reservoir plate and a multi-well tissue culture plate. 15A: “Type 1” riser configured to fit between flange of reservoir plate and flange of tissue culture plate. 15B: Shows riser 152 between culture plate 151 and liquid reservoir base plate 153.

FIGS. 16A-16B shows an example of a riser that is configured to fit between the bottom reservoir plate and multi-well tissue culture plate. 16A: “Type 2” riser, with upper extension configured for flange to rest on the extension, with upper extension acting as extension of the sidewall of the riser. 16B: Shows riser 162 between culture plate 161 and liquid reservoir base plate 163.

FIGS. 17A-17C show a side view comparison of stacked multi-well culture plate assemblies with and without risers. 17A: No riser. 17B: Type 1 riser. 17C: Type 2 riser.

FIGS. 18A-18D show an intestinal tissue model, as described in Example 7. 18A shows the different layers of the 3D intestinal tissue including the epithelial, fibroblast/collagen, and endothelial layers. 18B shows immunohistochemical staining for villin, a marker of brush border expressed on intestinal epithelial layer, 18C shows immunohistochemical staining for vimentin, a marker for fibroblasts; and 18D shows immunohistochemical staining for Von Willebrand factor, a marker for endothelial cells.

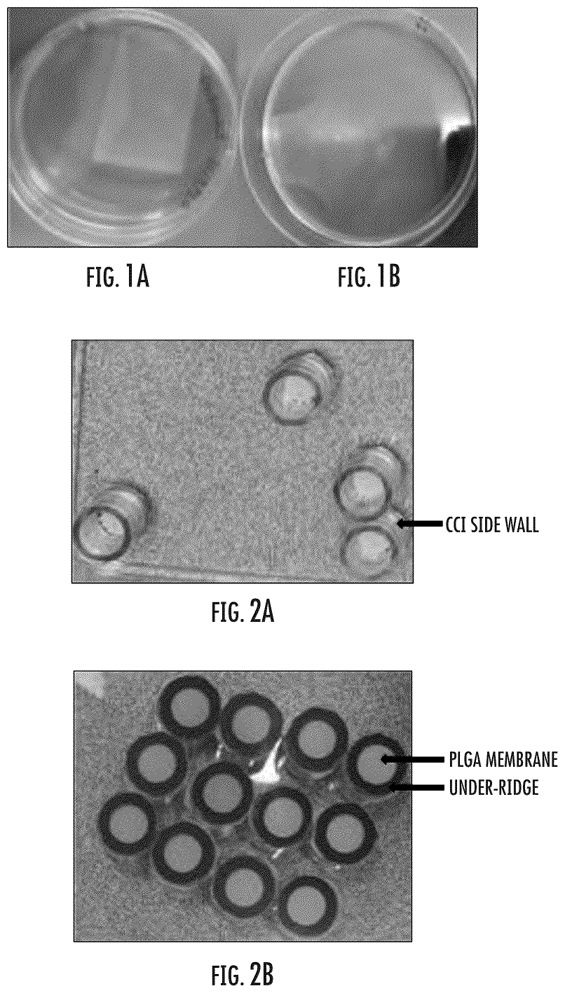

### DETAILED DESCRIPTION

The invention provides a stackable or nested tissue culture support system for growth of tissue culture cells and coculturing of two-or three-dimensional tissue models with multiple cell types. Stackable high-throughput multi-well plates are also provided. The stackable plates include at least one culture support plate that contains a microporous membrane, such as a biodegradable membrane, in the bottom of each well. In some embodiments, the wells of the top plate are configured to fit a second/middle plate that also contains a microporous membrane (i.e., wells of the top tissue culture support plate are configured to fit nested within, e.g., tapered to fit within, wells of a second tissue culture plate that is situated beneath the top tissue culture plate. In some embodiments, multiple (e.g., two, three, or more) stackable tissue culture plates are configured each with wells that fit nested within the plate that is directly below, wherein wells of each tissue culture plate contain a microporous membrane at the bottom of the well. The wells of the tissue culture support plate, or the wells of the bottommost tissue culture support plate in embodiments in which multiple stackable plates are deployed, are configured to fit inside of a bottom liquid reservoir plate. The liquid reservoir plate is configured to hold liquid medium either in individual wells or as a common liquid reservoir that is accessible by all wells of the tissue culture support plate that is immediately above the reservoir plate, with space between the bottoms of the wells of the tissue culture plate and the bottoms of the wells of a liquid reservoir plate that contains individual wells or the bottom of a liquid reservoir plate that contains a common liquid reservoir.

In some embodiments, two or more tissue culture support plates are included, with wells that are configured to nest (stack) within wells of the plate immediately beneath, with space between the porous membrane bottoms of the wells of each plate, and with the wells of the bottommost tissue culture support plate configured to nest within the wells of the bottom liquid reservoir plate, with space between the porous membrane bottoms of the wells of the bottommost tissue culture support plate and the bottoms of the wells of a liquid reservoir plate a liquid reservoir plate that contains individual wells or the bottom of a liquid reservoir plate that contains a common liquid reservoir. Also provided is a two-or three-dimensional tissue model that has been grown on a stackable tissue culture support as described herein.

Unless defined otherwise herein, all technical and scientific terms used herein have the same meaning as commonly understood by one of ordinary skill in the art to which this invention belongs. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this invention. Any methods and materials similar or equivalent to those described herein can be used in the practice or testing of the present invention.

Numeric ranges provided herein are inclusive of the numbers defining the range. Where a range of values is provided, it is understood that each intervening value, between the upper and lower limit of that range and any other stated or intervening value in that stated range is encompassed within the invention. The upper and lower limits of these smaller ranges may independently be included in smaller ranges, and are also encompassed within the invention, subject to any specifically excluded limit in the stated range. Where the stated range includes one or both of the limits, ranges excluding either or both of those included limits are also included in the invention.

### I. DEFINITIONS

“A,” “an” and “the” include plural references unless the context clearly dictates otherwise. Thus, for example, reference to “a cell” refers to one or more cells, and reference to “the system” includes reference to equivalent steps, methods and devices known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

An “apical” surface of a membrane is a surface that faces the external, e.g., gas phase, environment, e.g., air, for example the top surface of a membrane that is opposite of the bottom surface that faces a liquid culture medium.

A “basolateral” surface of a membrane is a surface that faces a liquid culture medium, for example, the bottom surface of a membrane that is opposite of the top surface that faces an external, e.g., gas phase, environment, such as air.

“Biodegradable” refers to biomaterials that are natural or synthetic in origin and are degraded in vivo or in vitro, either enzymatically or non-enzymatically or both, to produce biocompatible, toxicologically safe by-products which are further eliminated by normal metabolic pathways of cells (see, e.g., Makadia HK, Siegel SJ. Poly Lactic-co-Glycolic Acid (PLGA) as Biodegradable Controlled Drug Delivery Carrier. Polymers (Basel). 2011;3 (3): 1377-1397. doi: 10.3390/polym3031377).

As used herein, the term “co-culture,” and variations thereof, are used to specify growth and/or differentiation of two or more cell types or organ systems in direct or indirect contact with one another, generally (but not necessarily) at an air-liquid interface.

The term “differentiation medium” is used herein to refer to medium, e.g., culture medium, used for growth of cells at the air-liquid interface or in submerged culture (e.g., spheroids, organoids). The purpose of this medium is to induce the cells to organize, differentiate, and polarize to regenerate a three-dimensional in vitro tissue which mimics the in vivo tissue in structure and function. Differentiation medium may also be used to maintain the tissue in a differentiated state for an extended period of time.

As used herein, an “intestinal tissue culture model” or a “intestinal tissue equivalent” refers to human intestinal tissue models comprised of primary or non-primary human epithelial cells, along with fibroblasts, and optionally immune cells (e.g. macrophages, dendritic cells). The term intestinal cells includes enterocytes, Paneth cells, goblet cells, and enteroendocrine cells.

Unless otherwise stated, the term “medium” as used herein refers to both serum containing and serum free medium.

As used herein, a “microporous” membrane refers to a membrane that is a thin-walled structure having an open morphology of pore size, e.g., precisely controlled pore size, typically ranging from 0.03 μm (micrometer) up to 10 μm, or about 0.4 μm to about 8 μm, in diameter, e.g., mean pore diameter. Pores can be straight channel pores or tortuous path pores that allow for the selective passage of materials through the membrane. In terms of membrane geometry, three types of microporous membranes are typically used: flat sheet, hollow fiber, and tubular membrane. In some embodiments, the microporous membrane includes a flat sheet geometry. In other embodiments, the membrane includes hollow fiber or tubular geometry. In some embodiments, the microporous membrane (for example, PET or PC) includes straight channel pores. In other embodiments, the microporous membrane includes tortuous path pores (for example, PTFE).

The term “propagation medium” or “growth medium” is used herein to refer to medium, e.g., culture medium, used for growth of the cells in submerged culture. Propagation medium or growth medium, as the terms are used herein, may or may not include supplements which induce or support differentiation of cells in submerged culture, and therefore the use of the term is not restricted to use for cellular propagation absent any level of differentiation of the cells.

As used herein, a “respiratory tissue culture model”, “a respiratory tissue equivalent”, an “alveolar or an airway tissue culture model” or a “alveolar or airway tissue equivalent” are used interchangeably and refer to human tracheal or bronchial airway or alveolar tissue models comprised of primary or non-primary human epithelial cells, along with fibroblasts, and optionally immune cells (e.g. macrophages). The term alveolar cells includes alveolar epithelial cells (pneumocytes) of two subtypes: type I (the prevailing type) and type II alveolar cells. Type I alveolar cells are squamous extremely thin cells typically involved in the process of gas exchange between the lung and blood. A respiratory tissue culture model can include nasal cells, such as primary human nasal epithelial cells (HNEpC) which stain positive for cytokeratin. HNEpC cells can produce mucus, which binds particles that may be subsequently transported, typically to the pharynx by ciliary movement on the epithelial cells. HNEPC are useful for in vitro studies of these processes.

As used herein, the term “ridge” or “under-ridge” refers to an elevated part of a structure (e.g., an elevated crest) that protrudes from and surrounds the bottom of a well, i.e., surrounds the porous support membrane, e.g., surrounds the perimeter of the support membrane, on the side of the membrane that faces the medium reservoir when the support is in the upright (non-inverted) position, of a tissue culture support as described herein.

“Serum free medium” refers to culture medium which does not contain serum or a fractionated portion thereof. All components and amounts of serum free medium, in terms of their chemical composition, are defined and relatively pure by tissue culture standards of the art.

As used herein, a “tissue culture model”, “tissue culture equivalent” or a “tissue equivalent” refers to a well differentiated and polarized tissue regenerated by growing cells in a tissue culture vessel that allows cells to grow and interact with a surrounding extracellular framework in three dimensions to make an organ-like structure. This is in contrast with traditional two-dimensional cell cultures in which cells are grown in a flat monolayer on a plate.

### II. Nested Tissue Culture Support

### A. Stackable Support

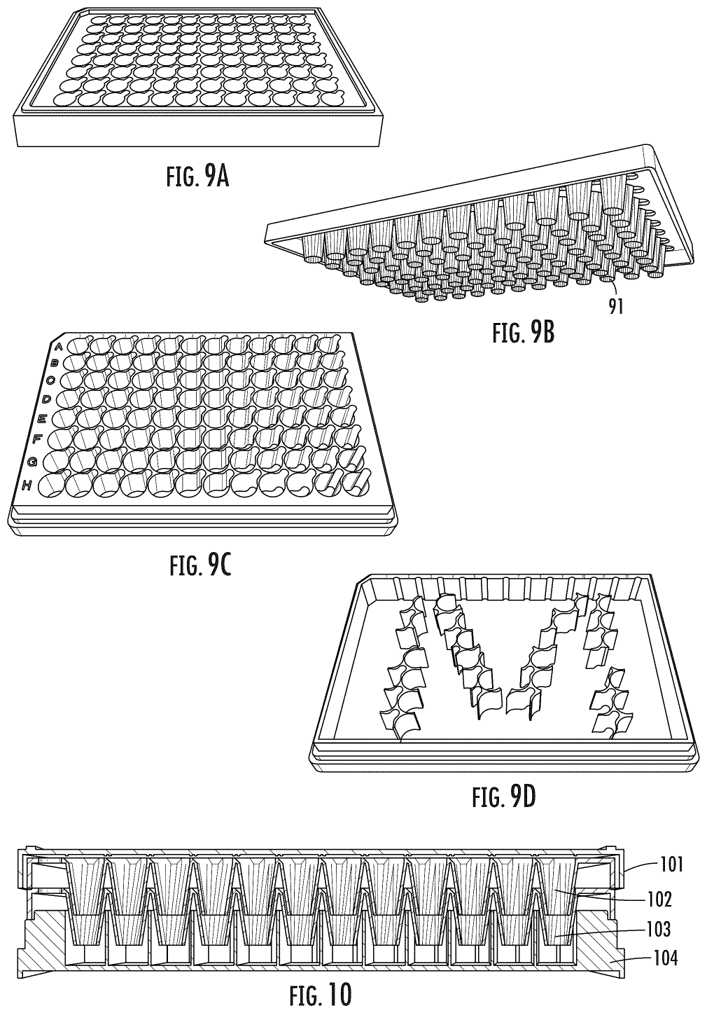

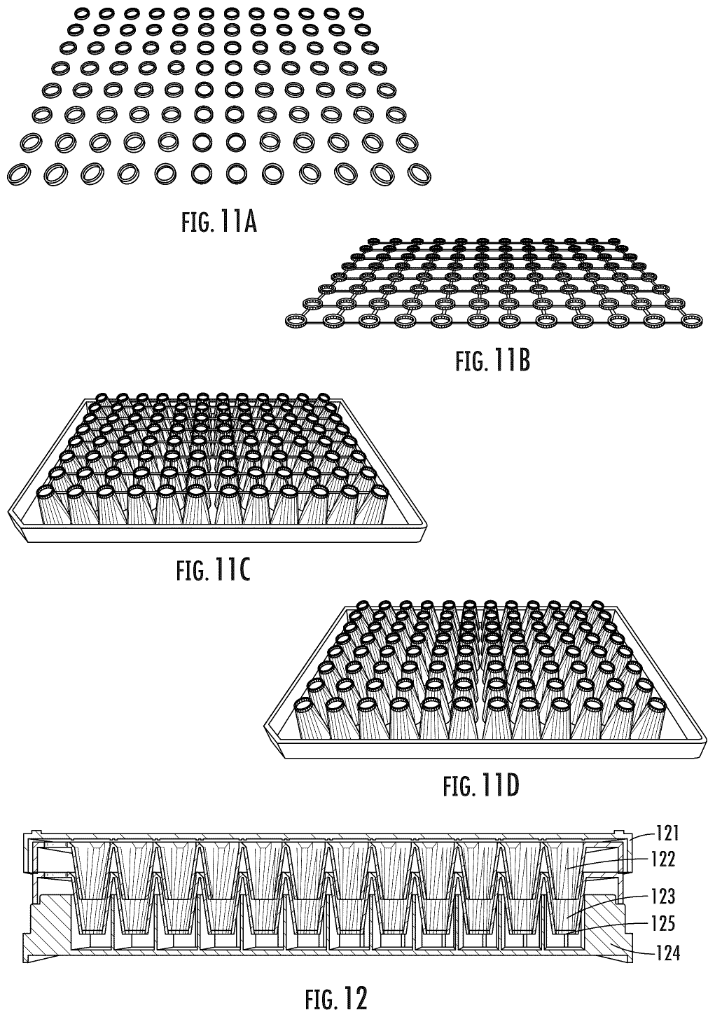

A stackable or nested tissue culture support device for growth of tissue culture, such as a two-dimensional or three-dimensional tissue model, is provided. The stackable support device includes: (a) a top cover; (b) a first well for growth of tissue culture cells, wherein at least a portion of the bottom of the first well is formed by a microporous, e.g., biodegradable, membrane material, and the top of the first well is open; and optionally, a second/middle well that also contains a microporous membrane, wherein the first well can fit inside of the second well with space between the membrane bottom of the first well and the membrane bottom of the second well; and (c) a bottom liquid reservoir that is configured to hold a liquid, e.g., liquid tissue culture differentiation or growth medium. wherein the liquid reservoir has a bottom that is located below the microporous membrane material at the bottom of the tissue culture support well. The liquid reservoir may contain either a well that is configured for the first well or optional second well to nest within (e.g., FIG. 9C), with space between the bottom of the tissue culture support well and the bottom of the reservoir, or may be a plate with dimensions that are larger than the tissue culture support well and not configured for the tissue culture support well to stack (nest) within (e.g., FIG. 9D). In some embodiments, the first well is configured to nest, i.e., fit within, a second well with spacing between the membrane bottom of the first well and the second well such that the first well is capable of holding a first tissue model or organ model and optionally, a second well is capable of holding a second tissue model or organ model under submerged condition, and then the first well, or second well if present, fits into a bottom well or plate which contains liquid or a tertiary organ model such as “lymph nodes” and also serves a liquid reservoir. One or more additional stacked wells (e.g., third, fourth, etc.) may be included in certain embodiments.

Each well may be tapered to fit within the well that is directly beneath, with a desired spacing between the bottoms of the nested wells, e.g., configured to permit a desired volume of liquid and/or cultured cells to be held in the space between the bottoms of the wells. Alternatively or additionally, a spacer may be provided between the wells, to support the well at a desired height above the well that is directly beneath or at a desired height above a bottom liquid reservoir plate. For example, a perimeter ring of a desired height may be used to separate and provide space between the membrane bottom of a well and the bottom of a well that is directly beneath or between a well and a bottom liquid reservoir plate.

In some embodiments, two or more stacked tissue culture supports are provided. For example, two tissue culture wells may be stacked on top of one another, each with a microporous membrane bottom. The tissue culture support wells stack and each nest inside of the well that is directly beneath, with space between the bottom of each well in the stack, and the bottommost tissue culture support well either stacks and nests inside of a bottom liquid reservoir well with space between the bottom of the bottommost tissue culture support well and the bottom of the liquid reservoir well, or the bottommost tissue culture support well is situated within a liquid reservoir plate with space between the bottom of the bottommost tissue culture support well and the bottom of the liquid reservoir plate.

In some embodiments, a biodegradable membrane is provided at the bottom of a tissue culture support well. Different cell types may be seeded on the top and bottom of the biodegradable membrane of tissue culture support well, and/or on different membranes of multiple tissue culture support wells in a stack. The biodegradable membrane allows cell to cell contact within the organ of a tissue model with multiple cell types or between organs in different stacked wells, allowing for cellular communication between the physiological systems.

The sides of the wells (and material between wells in multi-well plate embodiments) may be constructed, for example, of polystyrene, polycarbonate, copolyester, polypropylene, or other biocompatible plastic.

Typically, a top cover or plate lid is included in a stackable tissue culture device as described herein.

### B. Support Membrane

The porous base or support membrane must allow for passage of media from underneath the developing tissue. The support porosity must be of sufficient size to allow for passage of media and can be readily determined by the skilled practitioner. In some embodiments, the pore size (mean diameter) is about 0.4 μm to about 10 μm.

The support membrane may be constructed of an inert (e.g., non-biodegradable) material, such as polycarbonate, PET, or polytetrafluoroethylene, a biodegradable material (e.g., an electrospun nanofiber material), such as PLGA, PCA, or PCL, or a biological material such as collagen, chitosan, or glycosaminoglycan (GAG), or a combination thereof.

In some embodiments, the porous base may be a membranous base of polycarbonate, polyethylene terephthalate, or other culture compatible porous membrane such as membranes made of collagen, wettable fluoropolymers, cellulose, glass fiber or nylon attached to the bottom, on which the cells can be cultured. Examples of other suitable supports include, without limitation, an artificial membrane, an extracellular matrix component, a collagen gel, mixture or lattice, in vivo derived connective tissue, a mixed collagen fibroblast lattice, mixed extracellular matrix-fibroblast lattice, plastic, and a collagen sponge (U.S. Pat. No. 6,051,425).

### C. Under-Ridge

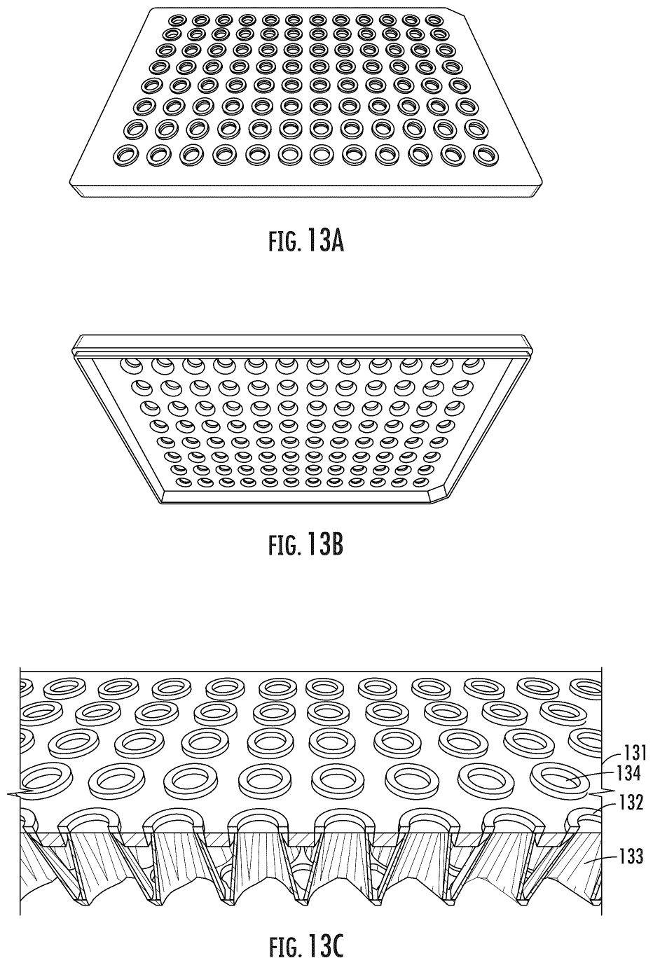

In some embodiments, an under-ridge, such as a containment insert or ring, is provided on the bottom of the well and surrounding the bottom of the membrane, configured to provide a volume in which cells may be seeded on the bottom surface of the membrane. The support device may be inverted to permit seeding of cells on the bottom surface of the membrane, and then turned right side up for seeding of cells on the top surface of the membrane. Examples of under-ridges are depicted in FIGS. 12 and 13.

In some embodiments, the under-ridge is 3-D printed or otherwise constructed as a ring or other shape that is sized to fit the bottom of the well (substantially the same shape and dimensions as the bottom of the well), and then affixed to the bottom side of the membrane, e.g., to the side of the membrane that is opposite of the side of the membrane that faces the top opening of the well. The under-ridge may be, for example, about 0.4 mm to about 2.5 mm in height, or about 0.9 mm.

The under-ridge may be, for example, constructed of PET, acrylonitrile butadiene styrene (ABS), polycarbonate (PC), polylactic acid (PLA), polystyrene (PS), or other extrudable or 3D printable polymer materials.

### D. Removable Plate

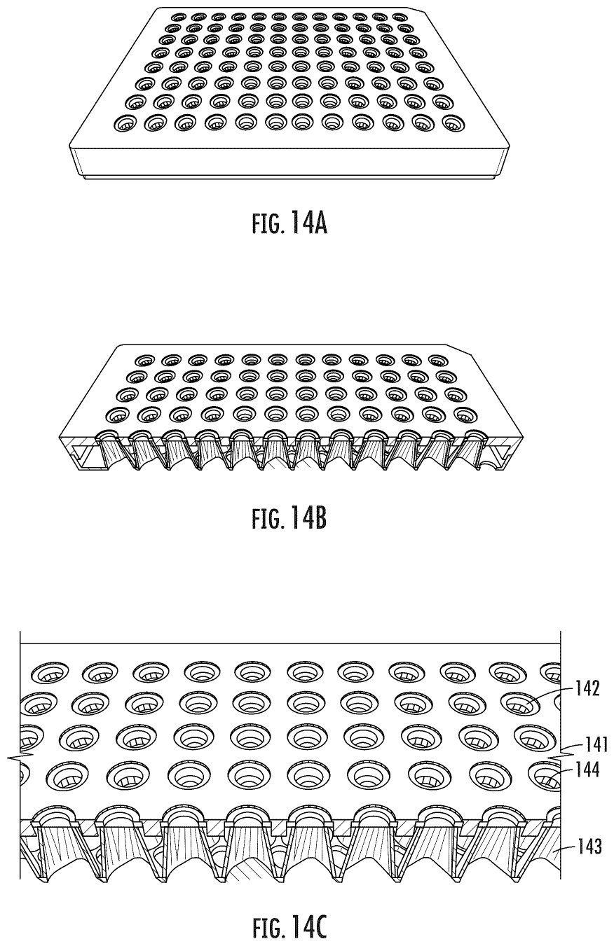

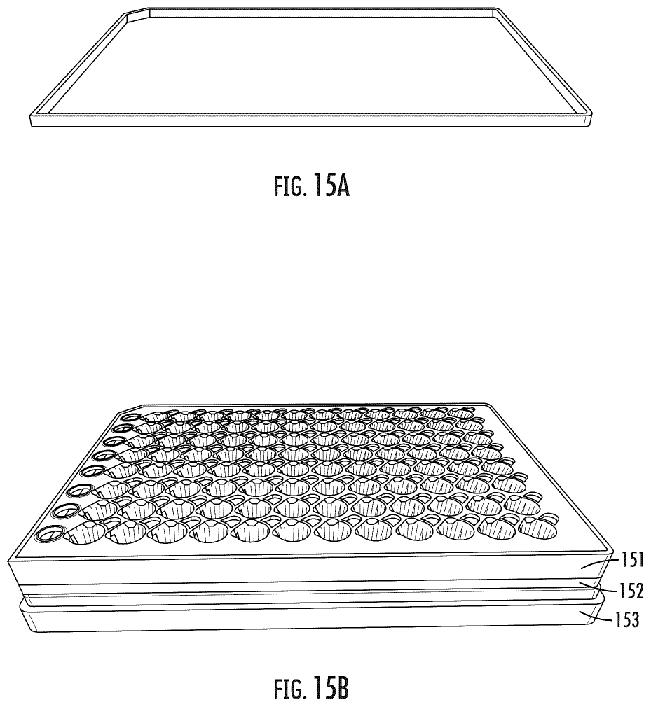

In some embodiments, a removable plate is used as a guide for seeding cells on the underside (basolateral side) of microporous membranes and for containment of cells and media on the basolateral side of the membrane in a stackable tissue culture support as described herein. The removable plate contains openings that are tapered such that the bottoms of the openings fit over and are substantially the same geometry and dimensions or smaller dimension than the microporous membrane at the bottoms of wells of an inverted stackable tissue culture support as described herein. The tapered openings may be used as a conduit for introduction of cells and media to the bottom surface of the microporous membrane, when the tissue culture support wells are inverted. The openings that are in contact with the inverted bottom of the membrane may include elements that provide an enclosure to contain cells and medium. Examples of removable plates for seeding of cells on the undersides of membranes are depicted in FIGS. 14A-14C and 15A-15C.

In one embodiment, the openings in the removable plate are fitted with a flexible, elastomeric material that provides tight contact or seal between the opening of the removable plate and the microporous membrane, thereby preventing leakage of liquid medium when cells are seeded on the underside of the membrane.

In another embodiment, the openings in the removable plate contain embedded o-rings that are substantially level with the microporous membrane. The o-rings provide a guide for seeding of cells and an enclosure to contain cells and medium.

### E. High-Throughput Device

In some embodiments, a stackable tissue culture support as described herein is configured with a plurality of nested wells (e.g., multi-well plate), e.g., for high throughput analysis. For example, the multi-well plate may contain, but is not limited to, any of 6, 12, 24, 48, 96, or 384 nested wells. In some embodiments, each well contains an under-ridge, as described herein.

For example, a multi-well high throughput format may be used to study drug safety and/or efficacy, environmental toxicity, disease treatment, organ-organ-interaction, or inflammation involving two or more organ systems, in a physiological tissue model.

### F. Risers

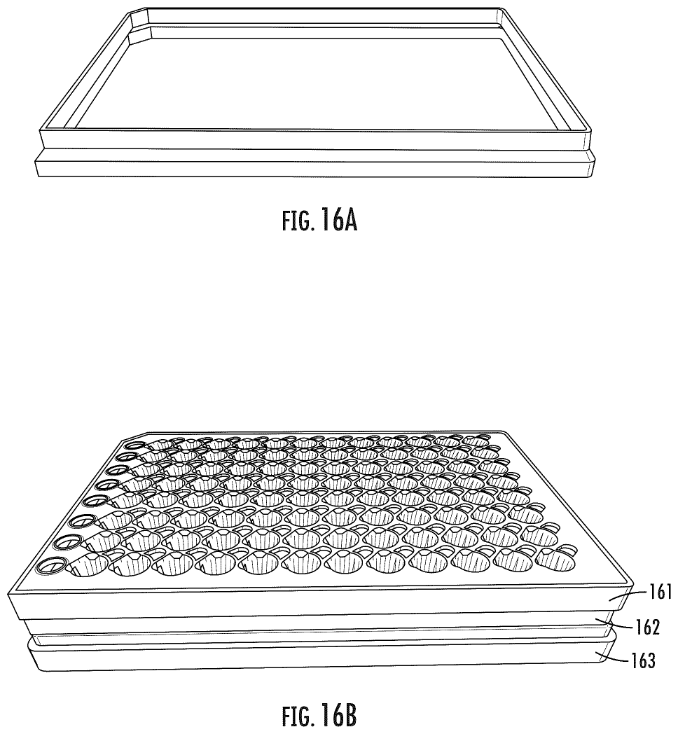

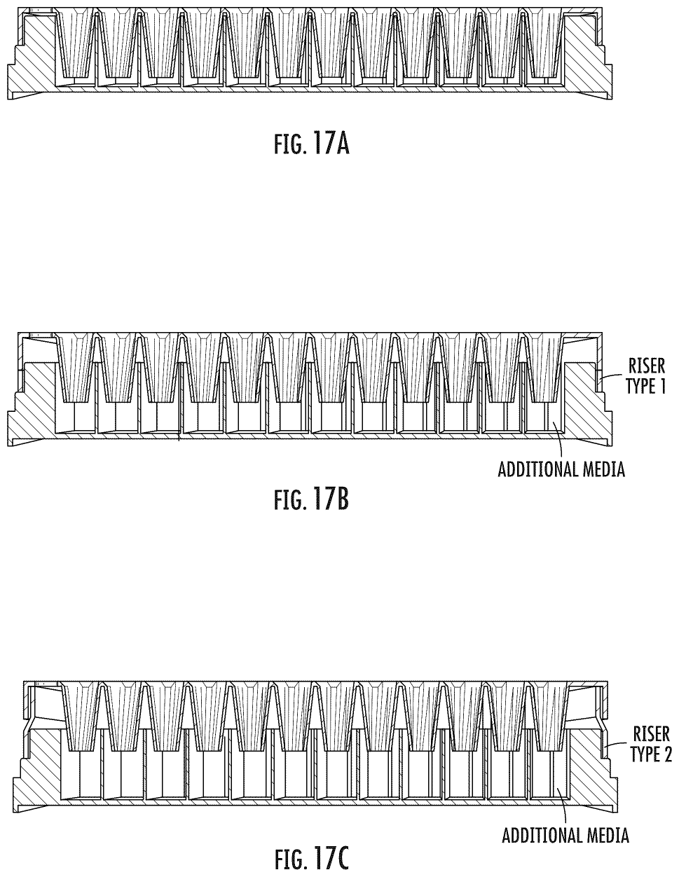

In some embodiments, risers are provided between culture supports and/or between a culture support and the bottom liquid reservoir. Examples of risers are depicted in FIGS. 15A-15B and 16A-16B. A riser may be in the form of a perimeter ring that fits around and provides a support to provide space between multi-well plates. For example, as depicted in FIGS. 15A-15B, the riser may fit between flange(s) around the perimeters of two multi-well plates and/or between a multi-well plate and a liquid reservoir plate. To create additional space between plates, an additional upper extension may be provided, as depicted in FIGS. 16A-16B, which is configured such that the flange of the upper plate rests on the extension.

### III. Three-Dimensional Tissue Models

Methods are provided for seeding and growing tissue culture cells on a stackable tissue culture support as described herein, thereby producing two-dimensional and/or three-dimensional tissue equivalents on one or more microporous culture support membrane(s) within the stacked support. In some embodiments, multiple cell types are seeded on biodegradable membrane(s) and as the membrane(s) degrade, the physiological systems come into contact with one another. Tissue equivalents grown in stackable tissue culture supports as described herein are also provided.

### A. Cells

In one embodiment, the cells which are seeded and grown on a tissue culture support as described herein are derived directly from in vivo tissue, referred to herein as primary cells. The cells may also be primary cells which have been passaged in culture, referred to herein as passaged primary cells. Passaged primary cells preferably still remain indistinguishable from the initially isolated primary cells, retaining their original characteristics, including growth parameters and inhibition, cell surface markers, biochemical response, and a finite life span in culture. The skilled artisan will recognize that primary cells derived from malignant tissue may not possess the characteristics of growth inhibition or a finite life span.

Primary cells may be obtained from either normal tissue or pathological tissue. Pathological tissue includes, without limitation, tissue wherein one or more of the cell types present are infected with a pathogen, cells that are derived from donors with a clinically defined disease condition, exhibit am abnormality in comparison to normal cells, or possess an acquired or inherited genetic defect, or are in some other way diseased.

An alternative to the use of primary or passaged primary cells is the use of immortalized or transformed cells. As is known in the art, immortalized cells are characterized as capable of multiple passaging in cell culture without undergoing senescence. Transformed cells share the characteristic of being immortalized, and in addition are not contact inhibited. One of skill in the art will recognize that an immortalized cell is not necessarily a transformed cell. As known in the art, non-transformed, non-immortalized cells can undergo only a finite number of passages in cell culture, at the end of which they undergo senescence, which is characterized as a loss of viability, and culminates in complete loss of the ability to propagate the cells in culture. Any combination of primary, passaged primary, transformed, and immortalized cells may be used to generate the tissue equivalent.

In some embodiments, the cells provided are originally isolated as primary cells, and then differentiated in culture to a desired phenotype prior to seeding. For example, this approach may be particularly useful in generating immune cells for use in producing the tissue equivalent.

Nonlimiting examples of cells that may be used for production of a tissue equivalent on a tissue culture support as described herein include epithelial (e.g., intestinal or airway (e.g., alveolar)) cells, fibroblasts, and immune cells (e.g. macrophages), and co-cultures thereof.

### B. Seeding

Cells are seeded under conditions appropriate for culture at the air-liquid interface. This involves seeding the cells onto a support which is conducive for growth of the cells at the air-liquid interface. One requirement for the support is that it is porous enough to allow passage of medium from below to the cells.

Seeding which is done prior to culture at the air-liquid interface is done by standard methods. This generally involves suspending the desired ratio and quantity of cells in liquid medium and depositing the cell-containing medium onto a support. The cells may be deposited into a well or an inverted well with an under-ridge, as described herein. Cells settle onto the porous support membrane at the bottom of the well or inverted well. Settling of the cells onto the support membrane after seeding is typically by gravity and takes anywhere from a few minutes to several hours. One of skill in the art can devise any number of other methods of depositing the cells onto the support, all of which are intended to be encompassed by the disclosure herein. In one embodiment, the amount (number) of cells seeded is about 1×10³ to about 1×10⁷ cells/cm². In another embodiment, the amount (number) of cells is about 1×10⁵ to about 1×10⁶ cells/cm².

Once the cells are deposited, the support is configured to allow culture medium to access the underside of the culture, while raising the seeded cells to the air-liquid interface.

At any interim post seeding of the cells onto the porous support, additional cells may be further seeded, either onto the support, or onto the growing/differentiating cells already present on the support. For example, additional seeding can be performed during culture or co-culture at the air-liquid interface by adding small quantities of medium from above which contains cells to be added onto the differentiating tissue culture. In one embodiment, immune cells are added onto the differentiating tissue culture.

The cells may also be manipulated prior to seeding onto the porous support. In one embodiment, fibroblasts are manipulated prior to seeding onto the porous support.

In one embodiment, cells, such as epithelial cells, are additionally cultured submerged in growth medium under conditions appropriate for cell propagation, prior to seeding onto the porous support. During this culture period they may optionally be cultured submerged under conditions appropriate for differentiation.

Cells may optionally be seeded on the underside of the porous support, i.e., on the side of the support membrane that faces the liquid medium when the support device is in the upright facing configuration, and then inverted prior to seeding on the top side of the porous support. The cells may be seeded onto the underside of the porous support by inverting the insert upside down. In some embodiments, the cells are manipulated prior to seeding on the underside of the porous support membrane. In one embodiment, the cells are suspended in cell growth medium and added to the underside of the membrane and allowed to attach to the membrane, which typically takes about 1 to about 24 hours. The volume of media added to culture the cells may be about 20 ml to about 500 ml. During this culture period, the cells may optionally be cultured submerged under conditions appropriate for a specific cell type, such as endothelial cells.

C. Culture at the air-Liquid Interface

Once seeded onto the support, the cells are raised to the air-liquid interface for culture under conditions appropriate for differentiation into the differentiated tissue equivalent. Methods for propagation and differentiation of cells at the air-liquid interface are well known in the art. The culture may be incubated, for instance, in a standard tissue-culture incubator under standard conditions. Conditions appropriate for differentiation into the tissue equivalent include temperature and content of the atmosphere in which the culture is incubated, media content, and optionally, the further seeding of additional cells onto the developing tissue. Typically, temperature and atmospheric conditions are about 37° C. in about 5% CO₂, although minor variations may be tolerated.

The period of culture at the air-liquid interface can extend from about 1 to about 120 days, although in some instances longer periods may be acceptable. A typical period of air-liquid interface co-culture is about 4 to about 14 days.

The amount of medium used can be as little as about 0.1 ml per cm² without any upper limit. In some embodiments, about 2.0 to about 10.0 ml of medium per cm² is fed to the developing tissue equivalent every other day. Flow through feeding for growth at the air-liquid interface may also be used. Flow through feed rates may be as little as 0.05 ml per cm² of culture tissue per day, or about 1.0 to about 5.0 ml per cm² of cultured tissue per day.

### D. Culture Media

The medium used for propagation and differentiation of the cells into the tissue equivalent of the present invention influences the properties of the final tissue equivalent product.

A variety of cell culture media known in the art are suitable for use as differentiation medium culturing of cells, i.e., culturing of one cell type or co-culturing of a multiplicity of cell types, at the air-liquid interface, the determination of which is within the ability of one of average skill in the art. In some embodiments, the differentiation medium includes a retinoid, such as retinoic acid, retinol, retinyl acetate, 13-cis retinoic acid, or 9-cis retinoic acid. In one embodiment, the medium includes about 10⁻⁵ to about 10⁻¹³ M of the retinoid, (e.g., about 5×10⁻¹⁰ M of a retinoid such as retinoic acid). In some embodiments, the concentration of the retinoid may be reduced incrementally over the period of co-culture. For example, the level of retinoic acid may be reduced from about 5×10⁻⁹ M down to about 5×10⁻¹³ M over the course of air-liquid interface culture period.

In some embodiments, the differentiation medium contains one or more of the following supplements: adenine, β-fibroblast growth factor, bovine pituitary extract, bovine serum albumin, calcium chloride, calf serum, carnitine, cholera toxin, adenosine monophosphate, endothelin-1, EGF (epidermal growth factor), epinephrine, estradiol, estrogen, ethanolamine, fetal bovine serum, FLT-3 (Fms-like tyrosine kinase 3), glucagon, granulocyte/macrophage-colony stimulating factor, hepatocyte growth factor, horse serum, human serum, hydrocortisone, insulin, insulin-like growth factor 1, insulin-like growth factor 2, isoproterenol, keratinocyte growth factor, linoleic acid,, newborn calf serum, nor-epinephrine, oleic acid, palmitic acid, phosphoethanolamine, progesterone, stem cell factor, transferrin, transforming growth factor-β1, triidothyronine, tumor necrosis factor a, vitamin A, vitamin B12, vitamin C, vitamin D, and vitamin E.

In one embodiment, the differentiation medium contains: DMEM (Dulbecco's Modified Eagle's Medium) and Ham's F12 medium at ratios such as 3:1 to 1:1, about 10% fetal calf serum, about 10 ng/ml epidermal growth factor, about 0.4 μg/ml hydrocortisone, about 1×10⁻⁶ M isoproterenol, about 5 μg/ml transferrin, about 2×10⁻⁹ M triiodothyronine, about 1.8×10⁻⁴ M adenine, about 5 μg/ml insulin, and about 1×10⁻⁶ M retinoic acid.

In another embodiment, the differentiation medium is serum free. Serum free medium may be made using basic media or components known in the art (e.g., DMEM, LHC9 (\ Part #12680013, ThermoFisher, Inc.), Ham's F12 medium, MEM (Modified Essential Medium), McCoy's 5A medium, MCDB 153 (Molecular Cell and Developmental Biology 153 medium) KGM (Keratinocyte growth Medium, Biowhittaker) EpiLife (Cascade Biologics, Inc.), or (normal human bronchial epithelial cell growth medium NHBE-GM). In one embodiment, the serum free medium is about a 3:1 ratio of DMEM: F12, supplemented with additional defined (non-serum) components, such as retinoic acid, or any of the other defined components described herein.

In one embodiment, the serum free differentiation medium contains about a 3:1 ratio of DMEM: F12, about 5×10⁻¹⁰ M retinoic acid, about 0.3 ng/ml keratinocyte growth factor, about 5 ng/ml EGF, about 0.4 μg/ml hydrocortisone, and about 5 μg/ml insulin. In another embodiment, the serum free differentiation medium is DMEM: F12 (about 3:1 ratio) containing retinoic acid (RA) at about 5×10⁻⁹ M, keratinocyte growth factor (KGF) at about 0.1 nM, about 0.4 μg/ml hydrocortisone, about 5 μg/ml insulin, SCF (about 2.5 ng/ml), GM-CSF (about 20 U/ml), TNF-a (about 0.25 ng/ml), and FLT-3 (about 2 ng/ml).

A variety of cell culture media known in the art is suitable for use as growth medium for cultivation or co-cultivation of cells in submerged medium. Examples of such media include, without limitation, DMEM, NHBE-GM, SFEM (serum free expansion medium), MEM, Medium 199, KGM, EpiLife, MCDB 153, and McCoy's 5A. In some embodiments, the growth medium for cultivation or co-cultivation of cells in submerged medium is serum free. One example of serum free growth medium which can be used is NHBE-GM containing SCF (about 25 ng/ml), GM-CSF (about 200 U/ml), TNF-α (about 2.5 ng/ml), and FLT-3 (about 20 ng/ml).

The determination of useful concentrations and combinations of the defined medium components or supplements described herein for use as growth medium or differentiation medium are within the ability of one of average skill in the art through no more than routine experimentation, as is the identification of additional supplements or medium components.

### EXAMPLES

The following examples are intended to illustrate, but not limit, the invention.

### Example 1. Generation of a Biodegradable Membrane for use in Tissue Culture Models

Electrospinning is a fiber production method which uses electric force to draw charged threads of polymer solutions or polymer melts up to fiber diameters in the order of several hundred nanometers. An electrospun poly (lactic-co-glycolide) (PLGA) membrane was produced having pore size <1 μm and thickness <10 μm, and biodegradable within 2-6 weeks. The membrane thickness and average pore size were determined to be 9.33 μm and 0.73 μm, respectively. The biodegradability of the membrane was characterized and biodegradation of the membrane determined to occur between 21 and 28 days, as determined by weight and tensile strength measurements.

Briefly, biodegradation experiments were designed to follow the American Society for Testing and Materials (ASTM) test method F1635-111, entitled: Standard Test Method for In Vitro

Degradation Testing of Hydrolytically Degradable Polymer Resins and Fabricated Forms for Surgical Implants. Biodegradation of the membranes was monitored by assessing tensile properties of the membranes.

A brief outline of the test protocol that was used is as follows:
1. PLGA electrospun samples are cut into rectangular pieces (˜12 mm×59 mm) and attached to clamps used for tensile strength experiments. The initial length (Lₒ) of the sample between the clamps and width of the sample is measured using to a digital caliper to ±0.5 mm.
2. Tensile strength measurements are made using an internally developed tensile strength meter. Samples are pulled at a constant rate of 17.8 mm/min and force measurements are taken every 0.237 seconds (252 measurements/minute).
3. Tensile strength measurements are made on the original (prior to biodegradation) membrane samples (n=4, time=0 days) and additional rectangular test samples are then fully immersed in the physiological solution (PBS, pH 7.4±0.2) and stored at 37±2° C.
4. After 7, 14, 21, 28, and 35 days of biodegradation in the PBS solutions, rectangular membrane samples (n=4) are removed. At each time period, the samples are removed and gently rinsed with sufficient distilled water to remove residual saline (PBS) on the surface of the membrane sample.
5. The samples are allowed to dry at room temperature for 48 hrs, after which they are clamped in place, and tensile measurements are made.

### Example 2. Biodegradation Profiles for Electrospun PLGA Biodegradable Membranes

Biodegradation data for the day 0, 7, 14, 21, and 28 day time points were collected. Since the Day 28 samples degraded to the point that accurate tensile strength measurements could not be made, the experiment was concluded on Day 28.

In the tensile strength experiments, the strain at breakage decreased to almost ˜25% of the original samples after 2 weeks of biodegradation and remained similar on Day 21; i.e., the PLGA membranes on Day 14 and Day 21 could be stretched considerably less than the original sample (prior to breakage). In addition, the membrane material became more difficult to break (increased tensile strength) and less flexible (increased Young's modulus) over the 3-week period. On Day 28, the membrane samples had degraded to the extent that only one of four samples could be mounted for tensile strength measurements. The other samples disintegrated or cracked during mounting between the 2 clamps (despite extra care being taken due to their fragile appearance). The tensile strength, the maximum strain at breakage, and Young's modulus for this single sample decreased to 16.5%, 15.1%, and 18.0%, respectively, of original (non-degraded) membranes. Thus, the Day 28 samples had degraded to the point where any physical handling of the samples caused them to disintegrate. Thus, it appeared that the PLGA membranes degraded between 21-28 days in PBS at 37° C.

### Example 3. Production of Single-Well Cell Culture Inserts (CCI) With PLGA Membrane and Under-Ridge

Single-well, cell culture inserts (CCI) were produced with PLGA electrospun membranes, and contained an under-ridge for seeding cells on the underside of the membrane. A top view is shown in FIG. 2A, and a bottom view, with the CCI inverted, is shown in FIG. 2B. The walls of the wells were constructed of copolyester and the bottom of the wells contained the PLGA membrane described in Examples 1 and 2.

PLGA membrane was attached to single well cell culture inserts (CCI). Under-ridges (thickness=0.9 mm, outer diameter (OD)=13 mm, inner diameter (ID=8.8 mm) were 3D printed of ABS material. These under-ridges were designed to be attached to the underside of individual cell culture inserts (CCI). Using a biocompatible adhesive, the under-ridges were attached to the bottom of the CCI. The under-ridge and the bottom side of the microporous PLGA membrane attached to the CCI to form a well that was 0.9 mm high and 8.8 mm wide which was used to seed cells onto the underside of the microporous membrane, thus providing a mechanism for growth of cells on the top and bottom surfaces of the membrane.

### Example 4. 96-Well Plate With Biodegradable Membrane and Under-Ridges

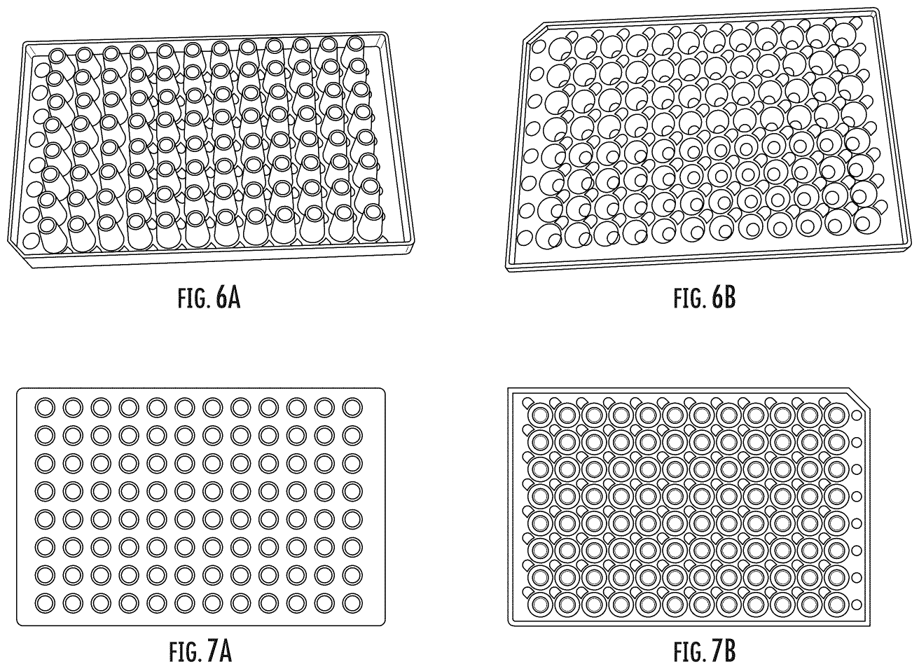

A mold was produced and 96-well plates were injection molded (polystyrene), with tapered wells and openings at the bottoms of the wells, as shown in FIGS. 6A-6B. Production of the plate proceeded as follows:

A sheet of PLGA membrane (82×124 mm) was attached to the molded piece using a biocompatible adhesive (FIG. 7A).

A Kkmoon (NEJE Master 2) 20W Laser Engraver, 5.5W Output was used to remove the excess membrane between the wells (FIG. 7B).

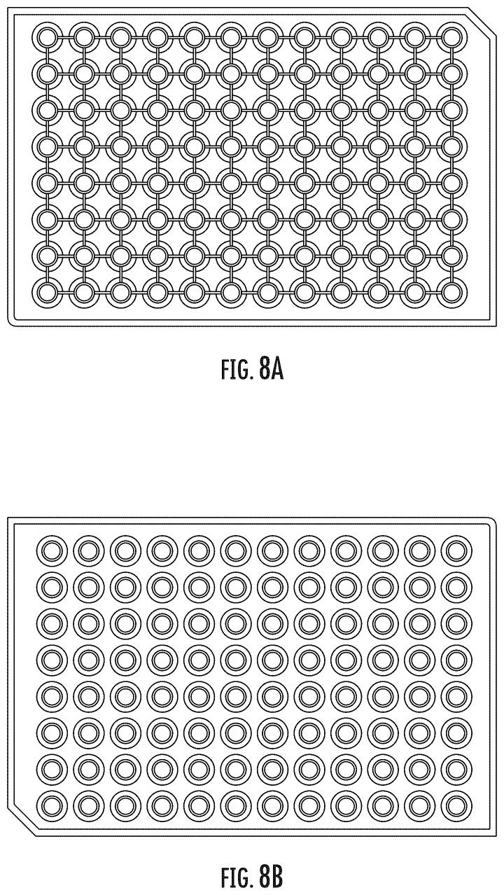

An interconnected sheet (matrix) of under-ridges 0.9 mm high was 3D printed (ABS) and attached to the under-side of the CCIP with biocompatible adhesive (FIG. 8A).

The interconnections between the under ridges were manually removed using a scalpel (FIG. 8B). Optimization of the thickness of the inter-connections should allow removal of both the excess membrane and the interconnections using a laser engraver.

### Example 5. Utility of 96-Well Plate With Biodegradable PLGA Membrane Over a 30 day Culture Period

Biocompatibility experiments were performed by using intestinal cells and fibroblasts to culture commercially available EpiIntestinal™ (MatTek Corporation; SMI-200-FT) tissue model on CCIs constructed as described in Example 3 and CCIPs constructed as described in Example 4. Standard protocols were used to produce the full thickness, EpiIntestinal™ tissue model. The commercially available SMI-200-FT model was also cultured using CCIs with a non-biodegradable, 0.4 μm polyethylene terephthalate (PET) microporous membrane for comparison. The tissues were cultured in commercially available cell culture media (MatTek Corporation; SMI-100-MM). All methods used for culture of the SMI-200-FT tissue on the PLGA CCIs were identical to those used for the PET CCIs, except as noted below.

### Growth of EpiIntestinal™ Tissue Model on Single-Well CCI and 96 Well CCIPs With PLGA Membrane

The procedure for growth of the tissue model on the PLGA and PET CCIs and CCIPs was as follows:

The PLGA CCIs and 96-well CCIPs were used without an extracellular matrix coating. The standard PET CCIs were collagen coated, as per standard production procedures.

The PLGA CCIs with the under-ridge were inverted and 75,000 endothelial cells in 150 μL of plating medium were seeded onto the bottom side of the PLGA membrane (seeding on the bottom side of the membrane was made possible by the under-ridge). For the PET CCIs, the CCIs remained upright and 75,000 endothelial cells were seeded into the CCI onto the face-up side of the membrane. PLGA 96-well CCIPs with the under-ridge were inverted and 15,000 endothelial cells in 30 μL of plating medium were seeded onto the bottom side of the PLGA membrane.

The PLGA and the PET CCIs seeded with the endothelial cells were incubated for 2 hr under “standard culture conditions” (37° C., 5% CO₂).

After 2 hr, the PLGA inserts containing endothelial cells were put into the upright position. A 400 μL seeding mixture of intestinal fibroblasts and epithelial cells in plating medium were added into both the PLGA and the PET CCIs.

The CCIs and CCIPs were cultured submerged (with 500 μL of plating medium on the basolateral side of the CCIs (underneath the membrane) and with the 400 μL of the seeding mixture on the upper side of the membrane overnight, under standard culture conditions. For the 96-well CCIPs, 80 μL of seeding mixture were added onto the upper side of the membrane.

The stackable 96-well CCIPs were cultured submerged (with 300 μL of plating medium/well on the basolateral side of the plate (underneath the membrane) and with the 80 μL of the seeding mixture on the upper side of the membrane overnight, under standard culture conditions.

After overnight culture under submerged conditions, the suprabasal medium was gently aspirated so that the suprabasal surface of the tissue was exposed to the atmosphere in the incubator. The tissues were fed from the basolateral side only using 5.0 ml of SMI-100-MM medium for CCIs and 275 ul/well for stackable CCIPs. This method is referred to as culturing at the air-liquid interface (ALI).

Tissues were cultured at the air liquid interface (ALI) under standard culture conditions for a total of 12 days. Over this time period, the SMI-100-MM medium was exchanged every other day with 5.0 mL or 275 ul of fresh SMI-100-MM medium.

On day 13 of the culture period (1 day submerged and 12 days at the ALI), tissues were fixed using 10% formalin, and standard histology procedures were used to produce hematoxylin & eosin (H&E) stained cross-sections of the tissues.

### Results

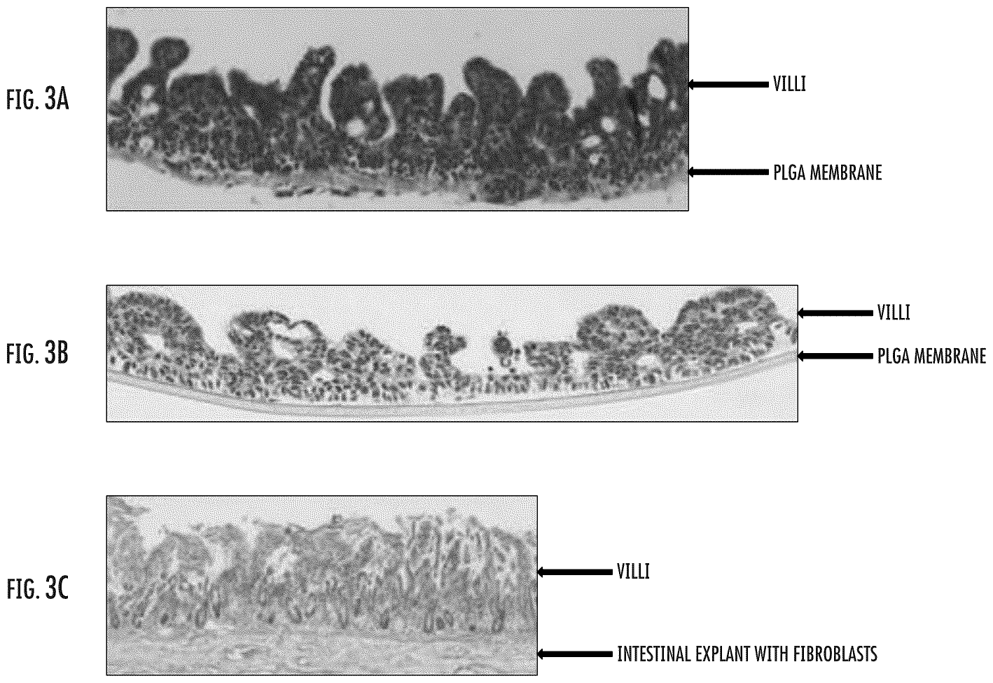

The histology of the EpiIntestinal™ tissues cultured on the biodegradable PLGA and the non-biodegradable PET membranes, along with a human small intestine explant, are shown in FIGS. 3A-3C. A well-differentiated, stratified intestinal tissue with well-developed villi was observed using the electrospun PLGA membrane CCIs with under-ridges (FIG. 3A). After the 13-day culture period, the three-dimensional tissue exhibited intestinal architecture with extensive villi on the suprabasal tissue surface, similar to the human explant tissue (FIG. 3C). Thus, in this 13 day experiment, the CCI inserts constructed with the PLGA membrane and the under-ridge are biocompatible. On the PET CCIs (FIG. 3B), villi length was shorter and the tissue structure was less compact.

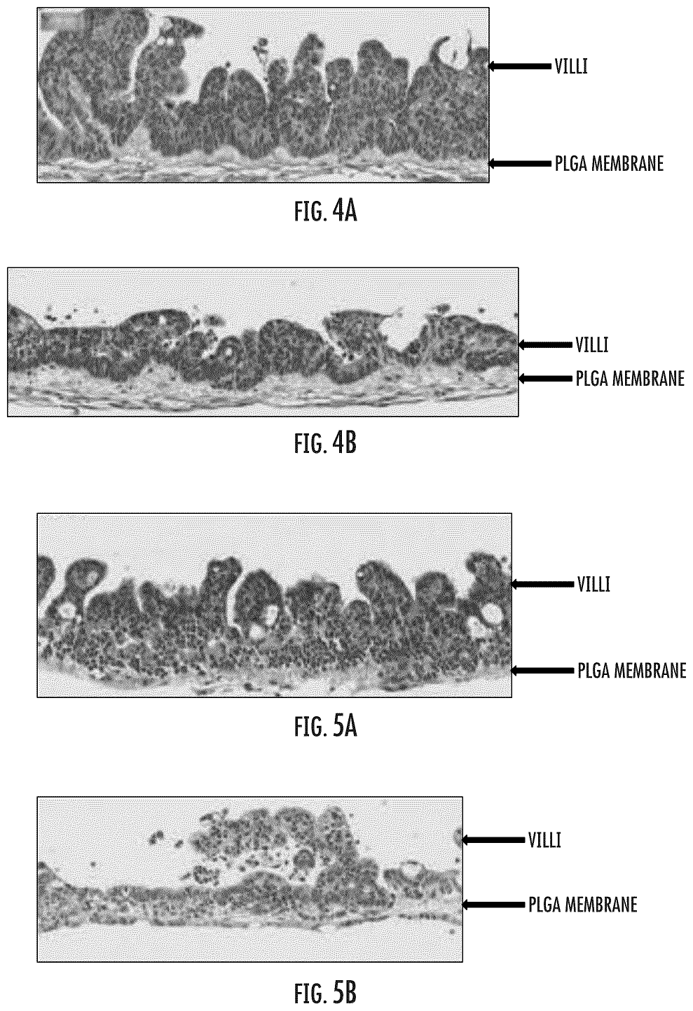

In culture experiments longer than 13 days, degradation of the PLGA membrane was observed microscopically in the H&E stained cross-sections. In the experiment shown in FIGS. 4A-4B, on Day 13 of the culture period (FIG. 4A), a continuous, compact membrane was observed. However, on Day 17 (FIG. 4B), the membrane appeared to be less well-defined, stretched/expanded, and it contained more vacuoles than on Day 13. In the experiment shown in FIGS. 5A-5B, on Day 13 (FIG. 5A), the membrane was continuous, albeit less well-defined than the membrane shown on Day 13 in FIG. 4A. On Day 21 (FIG. 5B), however, the membrane was observed only in isolated areas and had degraded to an extent such that, in some areas, only cells were observed beneath the basal tissue layer. For cultures extended beyond 21 days, none of tissues remained intact in the PLGA-containing CCIs. These culture results approximate the previous observations in the tensile strength experiments, as described in Example 2, although it appears that membrane degradation was slightly faster under culture conditions.

As the membrane started to degrade, seeded surface cells on top of the membrane and seeded bottom cells on the bottom of the membrane came into contact.

### Example 6. Fluorescent dye Compatibility of CCIs and CCIPs Constructed With PLGA Membrane

Experiments were performed to investigate the compatibility of CCIs and CCIPs assembled with PGLA membranes, as described in Example 3, with commonly used fluorescent dyes. Standard protocols were used to produce the full thickness intestinal tissue, SMI-200-FT (for detailed methods, see Example 4). The tissue was cultured for 7, 13, and 17 days. The tissues were fixed, sectioned, and stained with H&E to observe the tissue morphology.

Sections were deparaffinized and stained with the following fluorescently-labeled antibodies: a) fluorescein isothiocyanate (FITC)-conjugated antibody for cytokeratin (CK-19), an epithelial marker of the small intestine tissue (green), b) Alexa Fluor 555-conjugated antibody for vimentin, a marker for fibroblasts (red); and c) the nuclear stain 4′,6-diamidino-2-phenylindole (DAPI), a fluorescent stain that binds strongly to adenine-thymine-rich regions in DNA (blue).

The compatibility of the assembled CCI and CCIPs with commonly used fluorescent probes was demonstrated by immunostaining. All images were collected using a 10× objective on an Olympus FV1000 confocal microscope, with the following excitation wavelengths: DAPI: 405 nm; FITC: 473 nm, and Alexa Fluor 555, 561 nm. No autofluorescence/interference from the PLGA membrane was observed. Thus, standard immuno-staining techniques can be used tissue produced using the PLGA membranes.

An interesting observation resulted from a comparison between the H&E stained cross-sections and the immuno-stained cross-sections. Based on the Day 17 H&E stained cross-sections, it appeared that villi had degraded or had been lost, versus the Day 13 cultures. However, the immuno-stained cross-sections showed that cytokeratin-lined villi were still very plentiful on Day 17.

### Example 7. Growth of an Intestinal Tissue Model Containing Intestinal Endothelial, Fibroblasts, and Epithelial Cells on 96-Well Insert Plate With a Polycarbonate (PC) Membrane

An intestinal tissue model was prepared as follows:

1. A PC 96-well insert plate was used without an extracellular matrix coating.

2. 75,000 endothelial cells in 150 μL of plating medium were seeded into the wells of the PC 96-well insert plate (i.e., cells were seeded onto the apical membrane surface).

3. The PC 96-well insert plate seeded with the endothelial cells was incubated for 2 hours under standard culture conditions (37° C., 5% CO₂)

4. After 2 hours, a 400 μL seeding mixture of intestinal fibroblasts and epithelial cells in plating medium were added into the wells of the PC 96-well insert plate.

5. The PC 96-well insert plate was cultured submerged (with 300 μL of plating medium on the basolateral side of the membrane and with the 150 μL of seeding mixture on the apical side of membrane) overnight, under standard culture conditions.

6. After overnight culture under submerged conditions, the apical medium was gently aspirated. The tissues were fed from the basolateral side using 300 μL of SMI-100-MM (MatTek Corporation) and with 20 μL of SMI-100-MM on the apical side of the membrane. This method is termed culturing at the air-liquid interface (ALI).

7. Tissues were cultured at ALI under standard culture conditions for a total of 13 days. Over this period, the SMI-100-MM medium was exchanged every other day with 300 μL and 20 μL of fresh SMI-100-MM medium on the basolateral and apical sides of the membrane, respectively.

8. On day 14 of the culture period (1 day submerged and 13 days at the ALI), tissues were fixed using 10% formalin and standard histology procedures were used to produce hematoxylin & eosin (H&E) stained cross-sections of the tissues.

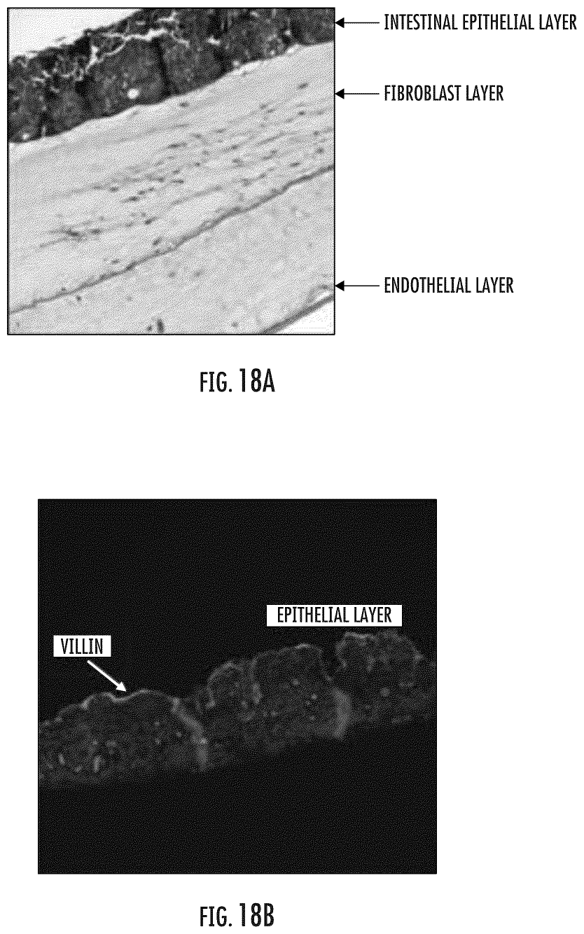

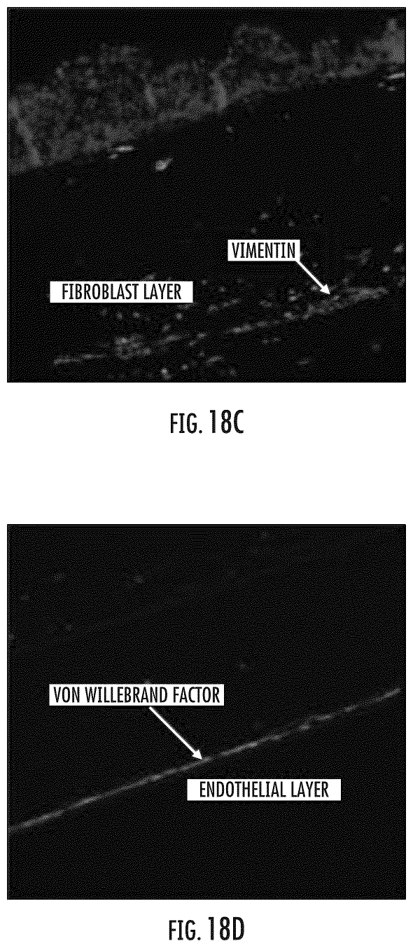

The results are shown in FIGS. 18A-18D.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention.

All publications, patents, and patent applications cited herein are hereby incorporated by reference in their entireties for all purposes and to the same extent as if each individual publication, patent, or patent application were specifically and individually indicated to be so incorporated by reference.

1. A stackable tissue culture support, comprising from top to bottom:
(a) a top cover;
(b) a culture support plate that comprises a plurality of wells, wherein the wells are open at the top and wherein at least a portion of the bottom of each of the wells comprises a microporous biodegradable membrane that is suitable for seeding of tissue culture cells; and
(c) a liquid reservoir plate, wherein the reservoir plate comprises individual wells configured such that the wells of the culture support plate nest within the wells of the reservoir plate, or wherein the reservoir plate comprises a common area for holding liquid to which the membranes at the bottoms of all wells of the culture support plate are accessible.

2. The stackable tissue culture support according to claim 1, further comprising:
one or more additional culture support plates, stacked above the culture support plate of (b) and beneath the top cover of (a),
wherein the one or more additional culture support plates each comprise the same number and arrangement of wells as the culture support plate of (b), and wherein the wells of each additional culture support plate are configured to nest within the wells of the culture support plate that is directly beneath, and
wherein the wells of each additional culture support plate are each open at the top and wherein at least a portion of the bottom of each of the wells of each additional culture support plate comprises a microporous membrane that suitable for seeding of tissue culture cells.

3. A stackable tissue culture support, comprising from top to bottom:
(a) a top cover; and
(b) two or more stacked culture support plates, wherein each of the stacked culture support plates comprises the same number and arrangement of wells as each of the other culture support plates in the stack, and wherein the wells of each culture support plate are configured to nest within the wells of the culture support plate that is directly beneath, and
wherein each culture support plate comprises a plurality of wells, wherein the wells are open at the top and wherein at least a portion of the bottom of each of the wells comprises a microporous membrane that is suitable for seeding of tissue culture cells; and
(c) a liquid reservoir plate, wherein the reservoir plate comprises individual wells configured such that the wells of the culture support plate directly above the reservoir plate nest within the wells of the reservoir plate, or wherein the reservoir plate comprises a common area for holding liquid to which the membranes at the bottoms of all wells of the culture support plate directly above the reservoir plate are accessible.

4. The stackable tissue culture support according to claim 1, wherein each well of the culture support plate(s) comprises an under-ridge around the bottom of the well that provides a bounded area for seeding of cells on the basolateral surface of the membrane when the plate is inverted, thereby providing an enclosure to contain cells and medium, and
wherein the under-ridges are separately attached underneath each well, or wherein the under-ridges are provided as an interconnected matrix under the wells of the plate with an under-ridge attached underneath each well.

5. A removable plate comprising openings that match the geometry of the wells of a culture support plate of claim 1,
wherein the openings comprise holes that are tapered to fit over the basolateral side of the microporous membranes when the culture support plate is inverted, and wherein the tapered openings provide a conduit for seeding of cells on the basolateral surface of the membrane and provide an enclosure to contain cells and medium, and
wherein the holes are fitted with a flexible, elastomeric material that provides a seal between each opening of the removable plate and the corresponding microporous membrane in the culture support plate, thereby preventing leakage of medium when cells are seeded on the undersides of the membranes.

6. A removable plate comprising openings that match the geometry of the wells of a culture support plate of claim 1,
wherein the o-rings are embedded in the holes such that when the removable plate is placed on top of an inverted culture support plate, the o-rings provide a guide for seeding of cells on the basolateral surfaces of the membranes and provide an enclosure to contain cells and medium.

7. The stackable tissue culture support according to claim 1, further comprising a riser between a culture support plate and the liquid reservoir plate, and/or between stacked culture support plates,
wherein the riser comprises material that is sufficient to separate the plates by a desired height, and wherein the riser comprise lengths corresponding to the lengths of the outer edges of the culture support plate and the liquid reservoir plate,
wherein the riser extends along the outer edge perimeter between the plates, thereby separating the plates by the desired height.

8. The stackable tissue culture support according to claim 2, wherein each well of the culture support plate(s) comprises an under-ridge around the bottom of the well that provides a bounded area for seeding of cells on the basolateral surface of the membrane when the plate is inverted, thereby providing an enclosure to contain cells and medium, and
wherein the under-ridges are separately attached underneath each well, or wherein the under-ridges are provided as an interconnected matrix under the wells of the plate with an under-ridge attached underneath each well.

9. A removable plate comprising openings that match the geometry of the wells of a culture support plate of claim 2,
wherein the openings comprise holes that are tapered to fit over the basolateral side of the microporous membranes when the culture support plate is inverted, and wherein the tapered openings provide a conduit for seeding of cells on the basolateral surface of the membrane and provide an enclosure to contain cells and medium, and
wherein the holes are fitted with a flexible, elastomeric material that provides a seal between each opening of the removable plate and the corresponding microporous membrane in the culture support plate, thereby preventing leakage of medium when cells are seeded on the undersides of the membranes.

10. A removable plate comprising openings that match the geometry of the wells of a culture support plate of claim 2,
wherein the o-rings are embedded in the holes such that when the removable plate is placed on top of an inverted culture support plate, the o-rings provide a guide for seeding of cells on the basolateral surfaces of the membranes and provide an enclosure to contain cells and medium.

11. The stackable tissue culture support according to claim 2, further comprising a riser between a culture support plate and the liquid reservoir plate, and/or between stacked culture support plates,
wherein the riser comprises material that is sufficient to separate the plates by a desired height, and wherein the riser comprise lengths corresponding to the lengths of the outer edges of the culture support plate and the liquid reservoir plate,
wherein the riser extends along the outer edge perimeter between the plates, thereby separating the plates by the desired height.

12. The stackable tissue culture support according to claim 3, wherein each well of the culture support plate(s) comprises an under-ridge around the bottom of the well that provides a bounded area for seeding of cells on the basolateral surface of the membrane when the plate is inverted, thereby providing an enclosure to contain cells and medium, and
wherein the under-ridges are separately attached underneath each well, or wherein the under-ridges are provided as an interconnected matrix under the wells of the plate with an under-ridge attached underneath each well.

13. A removable plate comprising openings that match the geometry of the wells of a culture support plate of claim 3,
wherein the openings comprise holes that are tapered to fit over the basolateral side of the microporous membranes when the culture support plate is inverted, and wherein the tapered openings provide a conduit for seeding of cells on the basolateral surface of the membrane and provide an enclosure to contain cells and medium, and
wherein the holes are fitted with a flexible, elastomeric material that provides a seal between each opening of the removable plate and the corresponding microporous membrane in the culture support plate, thereby preventing leakage of medium when cells are seeded on the undersides of the membranes.

14. A removable plate comprising openings that match the geometry of the wells of a culture support plate of claim 3,
wherein the o-rings are embedded in the holes such that when the removable plate is placed on top of an inverted culture support plate, the o-rings provide a guide for seeding of cells on the basolateral surfaces of the membranes and provide an enclosure to contain cells and medium.

15. The stackable tissue culture support according to claim 3, further comprising a riser between a culture support plate and the liquid reservoir plate, and/or between stacked culture support plates,
wherein the riser comprises material that is sufficient to separate the plates by a desired height, and wherein the riser comprise lengths corresponding to the lengths of the outer edges of the culture support plate and the liquid reservoir plate,
wherein the riser extends along the outer edge perimeter between the plates, thereby separating the plates by the desired height.
